(19) United States
(12) Patent Application Publication
(10) Pub. No.: US 2023/0126611 A1
(43) Pub. Date: Apr. 27, 2023

(54) INFORMATION PROCESSING APPARATUS, INFORMATION PROCESSING SYSTEM, AND INFORMATION PROCESSING METHOD

(71) Applicant: SONY GROUP CORPORATION, TOKYO (JP)

(72) Inventors: YUKI ITOTANI, TOKYO (JP); TETSUHARU FUKUSHIMA, TOKYO (JP); YOHEI KURODA, TOKYO (JP); KAZUTO YOKOYAMA, TOKYO (JP); KATSUFUMI SUGIMOTO, TOKYO (JP)

(21) Appl. No.: 17/906,280

(22) PCT Filed: Mar. 10, 2021
(86) PCT No.: PCT/JP2021/009444

§ 371 Date: Sep. 14, 2022

(30) Foreign Application Priority Data
Mar. 30, 2020 (JP) 2020-061407

(51) Int. Cl. A61B 90/00; A61B 34/20

(52) U.S. Cl. CPC A61B 90/361; A61B 34/20; A61B 2034/2068; A61B 2090/366

(57) ABSTRACT

To support easy arrangement of a robot, an information processing apparatus includes: a projection image generating section which generates a projection image that projects information specifying a range of motion of a target part of a medical arm onto the range of motion on the basis of first information regarding the range of motion and second information regarding a position and a posture of a projection device that projects an image in an operating room; and an output instruction section that outputs a projection instruction for the projection image to the projection device.

### TECHNICAL FIELD

The present disclosure relates to an information processing apparatus, an information processing system, and an information processing method.

### BACKGROUND ART

In surgery using a surgical support device such as a surgical robot, there is a problem of an operating room layout in which the surgical robot should be arranged. Determining the operating room layout is cumbersome and time consuming. One of the reasons for this is that surgical procedures or patient body shapes may vary, or operating room size or machines on hand may vary depending on the hospital. That is, the surgical environment has no reproducibility. Furthermore, as another reason, there is also a restriction that layout cannot be freely performed due to necessary arrangement of a plurality of other devices.

A number of techniques for supporting robot arrangement have been proposed. However, in order to automatically calculate robot arrangement, it is necessary to accurately grasp the operating room environment such as positions, sizes, and arrangement restrictions of people and other machines. Since appropriate arrangement of persons and other machines varies depending on surgical method, it is necessary to determine the operating room layout by actually incorporating the knowledge and judgment of medical staff such as doctors and nurses.

At present, a system including an operator console (master device) and a robot arm cart (slave device) that supports a surgical tool or a camera (endoscope, microscope, etc.) is widely used as a surgical robot. However, if an installer, such as a nurse, does not understand the overall range of motion of the surgical robot system (in particular, the robotic arm cart), appropriate arrangement and effective use of the range of motion is not possible. Furthermore, even in a case where a doctor actually arranges the robot arm cart directly, it is necessary to understand the range of motion before arranging the robot arm cart. In addition, not only the surgical robot as described above but also a surgical support device such as an articulated arm robot holding an endoscope has a similar problem in that it is important to arrange the surgical support device in consideration of a range of motion.

Patent Document 1 described below is a technique for automatically calculating a position of a robot, but it is necessary to accurately input or recognize a patient, a surgical procedure, and a surrounding environment into a system. Patent Document 2 described below is a system that supports arrangement of a robot in an operating room, but it is necessary to perform device tracking, and manual fine adjustment is difficult because a range of motion of the robot is not indicated.

### CITATION LIST

### Patent Document

Patent Document 1: Japanese Patent Application Laid-Open No. 2019-508134
Patent Document 2: Japanese Patent Application Laid-Open No. 2018-149321

### SUMMARY OF THE INVENTION

### Problems to be Solved by the Invention

The present disclosure provides an information processing apparatus, an information processing system, and an information processing method that support easy arrangement of a robot.

### Solutions to Problems

An information processing apparatus of the present disclosure includes:

a projection image generating section which generates a projection image that projects information specifying a range of motion of a target part of a medical arm onto the range of motion on the basis of first information regarding the range of motion and second information regarding a position and a posture of a projection device that projects an image in an operating room; and

an output instruction section that outputs a projection instruction for the projection image to the projection device.

An information processing system of the present disclosure includes:

a projection device which projects an image in an operating room;

a projection image generating section which generates a projection image that projects information specifying a range of motion of a target part of a medical arm onto the range of motion on the basis of first information regarding the range of motion and second information regarding a position and a posture of the projection device; and

an output instruction section which outputs a projection instruction for the projection image to the projection device.

An information processing method of the present disclosure

generates a projection image that projects information specifying a range of motion of a target part of a medical arm onto the range of motion on the basis of first information regarding the range of motion and second information regarding a position and a posture of a projection device that projects an image in an operating room, and

projects the projection image by the projection device.

### BRIEF DESCRIPTION OF DRAWINGS

FIG. 1 is a diagram broadly illustrating an example of a surgical system including an information processing system according to the present disclosure.

FIG. 2 is a diagram illustrating a state of surgery using the surgical system illustrated in FIG. 1.

FIG. 3 is a block diagram of an information processing system according to the present embodiment.

FIG. 4 is a diagram illustrating an example in which an image is projected onto a range of motion of a distal end portion by a projection device.

FIG. 5 is a plan view of another example in which an image is projected onto the range of motion of the distal end portion by the projection device.

FIG. 6 is a diagram illustrating an example in which each region of the image of the range of motion is colored.

FIG. 7 is a flowchart of an example of operation of the information processing system according to the present embodiment.

FIG. 8 is a diagram illustrating an example of a projection image in a case where the posture of the arm has been changed.

FIG. 9 is a block diagram of an information processing system according to a first variation.

FIG. 10 is a flowchart of an example of operation of the information processing system according to the first variation.

FIG. 11 is a diagram illustrating an example in which depth direction information is included in the image of the range of motion.

FIG. 12 is a diagram illustrating an example in which information for specifying the range of motion is aligned with an affected part in an affected part image.

FIG. 13 is a diagram illustrating an example in which composite information is projected from a projection device onto a floor surface of an operating room.

FIG. 14 is a flowchart of an example of operation of an information processing system according to a second variation.

FIG. 15 is a diagram broadly illustrating an example of a surgical system including an information processing system according to a third variation.

FIG. 16 is a block diagram of the information processing system according to the third variation.

FIG. 17 is a diagram illustrating an example in which an image is projected onto an integrated region.

FIG. 18 is a flowchart of an example of operation of the information processing system according to the third variation.

### MODE FOR CARRYING OUT THE INVENTION

Hereinafter, embodiments of the present disclosure will be described with reference to the drawings. In one or more embodiments illustrated in the present disclosure, elements included in each embodiment can be combined with each other, and the combined result also forms a part of the embodiments shown in the present disclosure.

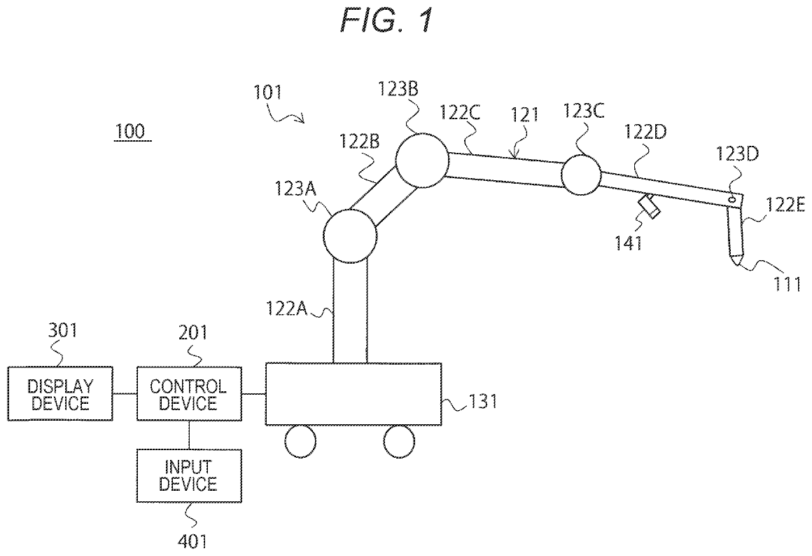

FIG. 1 is a diagram broadly illustrating an example of a surgical system 100 including an information processing system according to the present disclosure. The surgical system 100 includes a surgical robot (hereinafter, a robot) 101, a control device 201, a display device 301, and an input device 401. Note that in the following description, “user” refers to any medical staff who uses the surgical system 100, such as an operator or an assistant.

The robot 101 has a distal end portion 111 that performs an operation on an operation target, a medical arm (robot arm, articulated arm) 121 that supports the distal end portion 111 at a distal end, and a base 131 that supports a proximal end of the arm 121. The distal end portion 111 is an example of a movable target part in a medical arm.

Examples of the distal end portion 111 include a microscope section for enlarging and observing an observation target, an imaging device (camera, etc.) for capturing an observation target, a projection device (projector), an endoscope, forceps, tweezers, a pneumoperitoneum tube for pneumoperitoneum, and an energy treatment tool for performing incision of a tissue or sealing of a blood vessel by cauterization. The surgical system 100 may include a plurality of arms, for example, and each arm may be configured to have a different distal end portion 111. For example, the arms may be configured as an arm holding an imaging device, an arm holding forceps or tweezers, an arm having an energy treatment tool, or the like. Examples of the observation target include an observation part of a subject, specifically, a surgical site of a patient. The distal end portion 111 may include a plurality of the items listed here. By supporting an item using the distal end portion 111, the position of the item can be more stably fixed and a burden on the medical staff can be reduced as compared with a case where the medical staff manually supports the item.

One end of the arm 121 is attached to the base 131 such that the arm 121 extends from the base 131. The base 131 may be movable by a user on a floor surface using wheels attached to a lower portion. The user can fix the position of the robot by operating an unillustrated brake. The height of the arm 121 may be adjustable relative to the base 131.

The arm 121 includes a plurality of links 122A, 122B, 122C, 122D, and 122E and a plurality of joints 122A to 122D coupling the links 123A to 123E. The plurality of links 122A to 122E is mutually rotatable by the plurality of joints 123A to 123D. The distal end portion 111 is coupled to a distal end of the link 122E. When the distal end portion 111 is supported by the arm 121, the position and posture of the distal end portion 111 are controlled and stably fixed.

In the diagram, the configuration of the arm 121 is illustrated in a simplified manner for simplicity. In actuality, the shape, number, and arrangement of the joints 123A to 123D and the links 122A to 122E, the direction of the rotation axis of the joints 123A to 123D, a rotation or linear movement drive mechanism, and the like may be appropriately set so that the arm 121 has a desired degree of freedom. For example, the arm 121 may be suitably configured to have six or more degrees of freedom. Therefore, the distal end portion 111 can be freely moved within the movable scope of the arm 121.

The link 122D is provided with a projection device (projector) 141 that projects an image. The projection device 141 projects an image on the basis of a projection image provided from the control device 201. The projection device 141 may be coupled to the link 122D such that the projection direction of the projection device 141 can be rotated with a desired degree of freedom. Alternatively, the projection device 141 may be fixed to the link 122D such that the projection device 141 projects only in a specific direction. In a case where the projection device 141 is rotatable with a desired degree of freedom, the posture of the projection device 141 relative to the link 122D may be controllable by the control device 201. Parameters such as a focal length and a zoom magnification of the projection device 141 can also be controlled by the control device 201. The projection device 141 may be movable along the link 122D. In this case, the position of the projection device 141 on the link 122D may be controllable by the control device 201, or the position of the projection device 141 may be manually adjustable by the user. Examples of the target (projection target) on which the projection device 141 projects an image include a part (e.g., a surgical site) of a patient on a bed apparatus, a floor surface on which the bed apparatus is installed (a floor surface on which the bed apparatus is to be installed), and a lying surface of the patient in the bed apparatus (patient bed, operating table, etc.). The projection device 141 may be provided in a link other than the link 122D, or may be included in the distal end portion 111. Furthermore, the projection device 141 may be provided at any joint. In addition, the projection device 141 may be provided at a location other than the robot, such as a wall or a ceiling of the operating room.

The arm 121 is driven under the control of the control device 201. The joints 123A to 123D are provided with actuators including a drive mechanism such as a motor, an encoder that detects rotation angles of the joints 123A to 123D, and the like. The joints 123A to 123D are configured to be rotatable around a predetermined rotation axis by driving of the actuator. Then, the driving of each actuator is controlled by the control device 201, whereby the posture of the arm 121, that is, the position and posture of the distal end portion 111 are controlled. The control device 201 can grasp the current posture of the arm 121 and the current position and posture of the distal end portion 111 on the basis of information regarding the rotation angles of the joints 123A to 123D detected by the encoder. The base 131 may be equipped with a position detection function using a marker or the like. In this case, the control device 201 may acquire information on the position of the base 131 from the position detection function.

The control device 201 uses the grasped information regarding the position and posture of the arm 121 to calculate control values (e.g., rotation angles, generated torque, etc.) for the joints 123A to 123D to achieve movement of the distal end portion 111 according to operation input from the user. Then, the drive mechanisms of the joints 123A to 123D are driven according to the control values. The control method of the arm 121 by the control device 201 is not limited to a specific method, and various known control methods such as force control or position control may be applied.

As an example, when the user performs operation input via the input device 401, the driving of the arm 121 may be controlled by the control device 201, and the position and posture of the distal end portion 111 may be controlled. The control device 201 calculates control values (e.g., rotation angles, generated torque, etc.) for the joints 123A to 123D according to the operation input, and drives the drive mechanisms of the joints 123A to 123D according to the control values. After the distal end portion 111 has been moved to an arbitrary position, the distal end portion 111 is fixedly supported at the position after moving. Note that the arm 121 may be operated by a so-called master-slave method. In this case, the arm 121 may be remotely operated by the user via the input device 401 installed at a location in the operating room or a location away from the operating room.

The control device 201 integrally controls the operation of the surgical system 100 by controlling the operation of the robot 101 and the display device 301. For example, the control device 201 controls the driving of the arm 121 by operating the actuators of the joints 123A to 123D according to a predetermined control method. Furthermore, for example, the control device 201 generates image data for display by applying various types of signal processing to an image signal acquired by an imaging device included in the distal end portion 111 of the robot 101. The control device 201 also causes the display device 301 to display the generated image data. Examples of the signal processing include any of development processing (demosaic processing), image quality improvement processing (any of band emphasis processing, super resolution processing, noise reduction (NR) processing, and camera shake correction processing), enlargement processing (i.e., electronic zoom processing), and 3D image generation processing.

In addition, the control device 201 of the present embodiment calculates a range of motion (e.g., a range in which the distal end portion can move in a three-dimensional space) of a target part (e.g., the distal end portion of the arm) of the robot 101 in a three-dimensional space in the operating room, and generates a projection image that projects information (an image) specifying a motion space onto the range of motion. The control device 201 outputs a projection instruction for the generated projection image to the projection device 141. The projection device 141 projects the projection image provided from the control device 201. The user can intuitively grasp the range of motion of the target part of the robot 101 by viewing the projected image. A configuration in which the control device 201 generates a projection image and a configuration in which the projection device projects the projection image will be described later. The target part may be an arbitrary part such as an arbitrary link or an arbitrary joint of the arm 121 other than the distal end portion 111.

Transmission and reception of information between the control device 201 and the distal end portion 111, transmission and reception of information between the control device 201 and the joints 123A to 123D, and transmission and reception of information between the control device 201 and the projection device 141 are performed by wired communication or wireless communication. Wired communication may be communication by an electric signal or communication by an optical signal. As a transmission cable used for wired communication, an electric signal cable, an optical fiber, or a composite cable of the foregoing is used according to the communication method. A wireless communication method may be an arbitrary method such as a wireless local area network (LAN), Bluetooth, a dedicated communication method, 4G communication, or 5G communication. In the case of wireless communication, since it is not necessary to lay a transmission cable, a situation may be eliminated in which movement of the medical staff in the operating room is hindered by a transmission cable.

The control device 201 may be a processor such as a central processing unit (CPU) or a graphics processing unit (GPU), a microcomputer in which a processor and a storage element such as memory are combined, a control board, or the like. The processor of the control device 201 operates according to a predetermined program, whereby the above-described various functions may be achieved. Note that in the illustrated example, the control device 201 is provided as a separate device from the robot 101, but the control device 201 may be installed inside the base 131 of the robot 101 and configured integrally with the robot 101. Alternatively, the control device 201 may be configured by a plurality of devices. For example, a microcomputer, a control board, or the like may be disposed in each of the distal end portion 111 and the joints 123A to 123D of the arm 121, and these elements may be communicably connected to each other to achieve similar function to the control device 201.

As an example, the display device 301 is provided in an operating room, and displays an image corresponding to image data generated by the control device 201 under the control of the control device 201. The display device 301 is a display device such as a liquid-crystal display device or an electroluminescent (EL) display device, for example. The display device 301 displays an image of a surgical site captured by an imaging device provided in the distal end portion 111, another part of the robot 101, the operating room, or the like, or an image of the environment or equipment in the operating room. The display device 301 may display various types of information regarding a surgery instead of or together with images of the surgical site, environment, equipment, or the like. Examples of the information include body information of the patient or information regarding a surgical procedure of the surgery. The display device 301 may be provided in a plurality thereof. A plurality of imaging devices may be provided, and image data obtained by every imaging device may be displayed on different display devices. Image data captured by the plurality of imaging devices may be simultaneously displayed on the same display device.

The input device 401 is an operation device for the user to perform various operation input. The input device 401 is a device that can be operated even if the user has a surgical tool in hand, such as a foot switch or a device that performs voice recognition as an example. Alternatively, the input device 401 may be a device capable of accepting operation input in a non-contact manner on the basis of gesture detection or line-of-sight detection using a wearable device or a camera provided in the operating room. Furthermore, the input device 401 may be a device manually operated by the user, such as a touch panel, a keyboard or a mouse, or a haptic device. In addition, in the case of a master-slave type surgical system, the input device 401 is an input device included in a master console and operated by an operator.

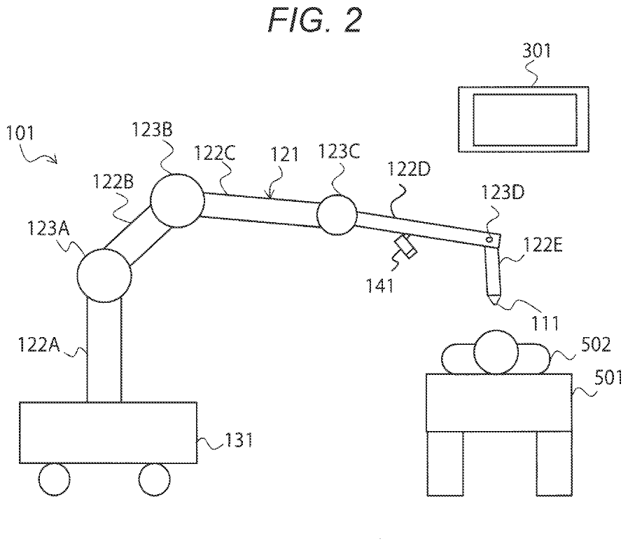

FIG. 2 is a diagram illustrating a state of surgery using the surgical system 100 illustrated in FIG. 1. FIG. 2 broadly illustrates a state in which an unillustrated user (herein, an operator) is performing a surgery on a patient 502 on a bed apparatus 501 using the surgical system 100. Note that in FIG. 2, for simplicity, illustration of the control device in the configuration of the surgical system 100 is omitted, and the robot 101 is illustrated in a simplified manner.

During surgery as illustrated in FIG. 2, an image of a surgical site captured by the robot 101 is enlarged and displayed on the display device 301 in the operating room using the surgical system 100. The user may observe the state of the surgical site through a video projected on the display device 301. The user may perform treatment by directly holding the surgical tool at the side of the patient 502, or may perform treatment by remotely operating the distal end portion 111 via the input device 401 through a master-slave method. In this case, an arm provided with the imaging device and an arm holding the surgical tool may be separate arms. Furthermore, the projection device 141 may be provided in the same arm as one of the arm provided with the imaging device and the arm holding the surgical tool, or may be provided in a third arm different from those arms. By providing the projection device 141 on at least one of the arms, an image may be projected from the projection device 141 onto the range of motion of the distal end portion 111 during surgery, and the user may perform surgery while confirming the range of motion of the distal end portion 111. A state in which an image is projected onto the range of motion may be displayed on the display device 301. In this case, the user can confirm the range of motion while viewing the display device 301. The user can intuitively grasp the range of motion of the distal end portion 111 from the projection image in both a case where surgery is performed at the side of the patient and a case where surgery is performed remotely. The user can perform various treatments such as excision of an affected part while confirming the range of motion of the distal end portion 111. Here, an example in which an image is projected from the projection device 141 during surgery has been described, but it is also possible to project an image in a preparation stage before surgery to perform tasks such as positioning of the bed apparatus or positioning of a subject on the lying surface of the bed apparatus. Details of these examples will be described later.

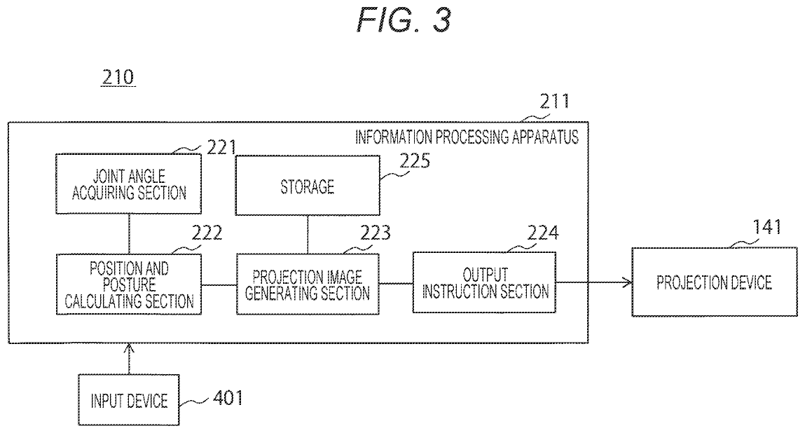

FIG. 3 is a block diagram of an information processing system according to the present embodiment. An information processing system 210 is configured to use the control device 201, the projection device 141, and the input device 401 in the surgical system 100 of FIG. 1.

The information processing system 210 includes an information processing apparatus 211, a projection device 141, and an input device 401. The information processing apparatus 211 includes a joint angle acquiring section 221, a position and posture calculating section 222, a projection image generating section 223, an output instruction section 224, and storage 225. The joint angle acquiring section 221 acquires information on the joint angles (rotation angles) of the joints 123A to 123D from the encoders provided in the joints 123A to 123D. The information on the joint angles of the joints 123A to 123D may be stored in the storage 225 of the control device 201 in advance. In this case, information on the joint angles of the joints 123A to 123D may be acquired from the storage 225.

The position and posture calculating section 222 calculates the position and posture of the projection device 141 on the basis of the joint angles (arm postures) of the joints connecting the links present between the base 131 and the location where the projection device 141 is provided. In the present example, since the projection device 141 is installed in the link 122D, the position and posture of the projection device 141 are calculated on the basis of the joint angles of the joints 123A to 123C. In a case where the projection device 141 is relatively rotatable with respect to the link 122D with an arbitrary degree of freedom, a relative posture of the projection device 141 with respect to the link 122D is specified, and the posture of the projection device 141 is calculated on the basis of the posture of the arm 121 and the relative posture. The posture of the projection device 141 can be represented by three angle variables in a three-axis space, for example. The position of the projection device 141 can also be represented by coordinates in a three-axis space. In a case where the position of the projection device 141 is movable (e.g., in a case where the position is movable in parallel along the link 122D), the position of the projection device 141 need only be calculated on the basis of the relative position of the projection device 141 in the link 122D and the posture of the arm.

The projection image generating section 223 specifies a range of motion of the target part of the robot 101 relative to the base 131 (refer to FIG. 1). The target part is the distal end portion 111 in the present embodiment. The range of motion of the distal end portion 111 relative to the base 131 may be specified in advance from items such as robot design information, a result of robot operation confirmation performed in advance by a test, or a simulation. The range of motion of the distal end portion 111 is a relationship that is fixed relative to the base 131. Information regarding the range of motion of the distal end portion 111 relative to the base 131 is stored in the storage 225. The projection image generating section 223 acquires the information regarding the range of motion of the distal end portion 111 relative to the base 131 by reading from the storage 225. When the target part is a part other than the distal end portion 111, the range of motion information of the part is stored in the storage 225. The storage 225 is an arbitrary storage device that stores data, such as memory, a hard disk, a solid-state disk (SSD), or an optical recording medium. In a case where the height of the arm 121 is adjustable relative to the base 131, information regarding the range of motion of the distal end portion 111 may be stored in the storage 225 for every height. Alternatively, the range of motion may be specified by adding an offset according to the height.

On the basis of the range of motion information of the distal end portion 111 relative to the base 131 and the position and posture of the projection device 141, the projection image generating section 223 generates a projection image that projects information (an image) specifying the range of motion of the distal end portion 111 onto the range of motion. That is, by projecting the projection image from the projection device 141, an image capable of identifying the range of motion of the distal end portion 111 is displayed in the range of motion. In generating the projection image, parameter information (focal length, zoom magnification, etc.) of the projection device 141 may be used in addition to the position and posture of the projection device 141.

The projection target of an image of a motion space is an observation part (e.g., a surgical site) of a patient allowed to lie on a bed apparatus, a lying surface of the patient in the bed apparatus, or a floor surface on which the bed apparatus is installed (a floor surface on which the bed apparatus is to be installed), for example.

In a case where the image of the motion space is projected on the surgical site of the patient, the user (operator) can grasp the range of motion of the distal end portion 111 during surgery while maintaining a line of sight from the surgical site. Furthermore, in a case where the image of the range of motion is projected on the lying surface of the patient in the bed apparatus, it is easy to allow the patient to lie on the bed apparatus such that the surgical site of the patient is positioned in the range of motion. In addition, in a case where the image of the range of motion is displayed on the floor surface, it is possible to easily perform positioning of the bed apparatus on which the patient is allowed to lie or positioning of the robot 101 or the arm 121.

The output instruction section 224 outputs a projection instruction for the projection image generated by the projection image generating section 223 to the projection device 141. The projection device 141 projects a projection image in accordance with the projection instruction from the output instruction section 224. The information processing apparatus 211 may control the position or posture of the projection device 141 relative to the link 122D to a predetermined position or posture according to the posture of the arm 121 (joint angle of each joint). As a result, regardless of the posture of the arm 121, projection can be appropriately performed in the range of motion of the projection device 141 even when the motion space is positioned outside the projectable range.

The projection device 141 is a two-dimensional projection device (2D projector) that projects a two-dimensional image or a three-dimensional projection device (3D projector) that projects a three-dimensional image. The projection image generating section 223 generates a projection image adapted to each method depending on whether the projection device 141 is a two-dimensional projection device or a three-dimensional projection device. In the case of a three-dimensional projection device, by projecting a three-dimensional image from the projection device 141, the range of motion of the distal end portion 111 is stereoscopically displayed in three-dimensional space, and the range of motion can be intuitively recognized up to the depth direction. In the case of a two-dimensional projection image, a two-dimensional image is projected from the projection device 141. As an example, an image of a region at an arbitrary height in the range of motion is displayed as the two-dimensional image. The height in the range of motion at which the range of motion is to be displayed may be determined, and a projection image that projects the range of motion having the determined height may be generated.

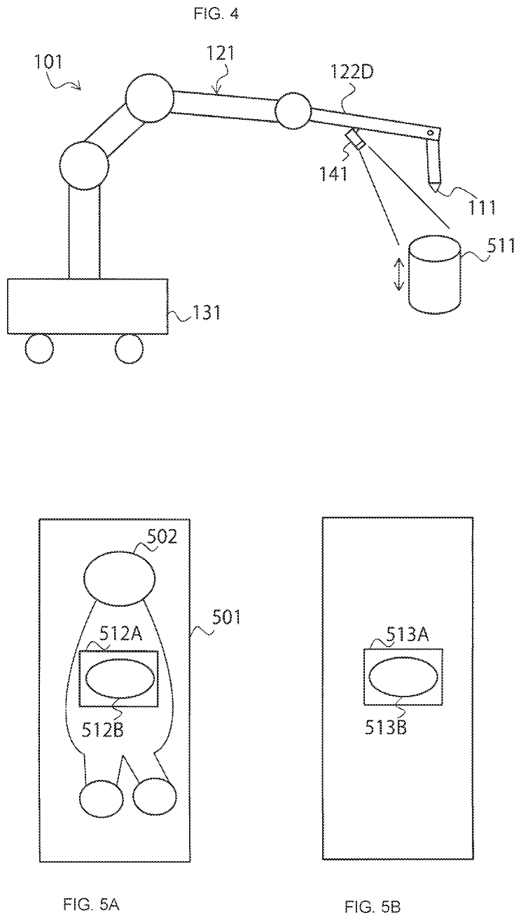

FIG. 4 illustrates an example in which the range of motion of the distal end portion 111 is projected by the projection device 141 through a three-dimensional image. The range of motion is perceived in three dimensions through a projection image 511 representing the three-dimensional range of motion generated on the basis of the position of the user or the position of a photograph device. As a method for allowing the range of motion to be perceived in three dimensions, the user may wear dedicated glasses (e.g., including two lenses) and two two-dimensional images may be simultaneously projected from the projection device 141 to cause parallax through the dedicated glasses, thereby allowing the range of motion to be perceived in three dimensions (passive method). Alternatively, the user may wear dedicated eyeglasses (e.g., including one lens) and different images may be alternately projected from left and right at a high speed from the projection device 141, thereby allowing the range of motion to be perceived in three dimensions (frame sequential method). The range of motion may be perceived in three dimensions through a method other than those described here. The distal end portion 111 can move within the range of motion. The range of motion can be arbitrarily defined according to the configuration of the distal end portion 111. For example, in a case where the distal end portion 111 includes an imaging device, the range of motion may be a region that can be captured by the imaging device (region where an image can be acquired). In a case where the distal end portion 111 includes a surgical tool, the range of motion may be a region where the surgical tool can be moved or a region where the surgical tool can be appropriately utilized on an operation target (surgical site, etc.).

FIG. 5(A) is a plan view of an example in which the range of motion of the distal end portion 111 is projected by the projection device 141 through a two-dimensional image. It is assumed that the patient 502 is lying on the bed apparatus 501 and is undergoing surgery by a user (operator) using the robot 101 as illustrated in FIG. 2 described above. A two-dimensional projection image 512A includes a range of motion image 512B. The image 512B and the projection image 512A are displayed in different modes, for example, and can be easily distinguished. For example, the image 512B and the projection image 512A are displayed in different colors or in different patterns. The positioning of the bed apparatus 501 and the positioning of the patient 502 in the bed apparatus 501 are performed such that the surgical site of the patient is positioned in the range of motion. Furthermore, since the range of motion is displayed as an image in accordance with the surgical site, the user can confirm the range of motion of the distal end portion 111 while maintaining a line of sight from the surgical site.

As an example, the displayed range of motion is a range of motion in a plane at the height of the patient 502 in the three-dimensional range of motion of the distal end portion 111. Information on the posture (height, inclination, etc.) of the bed apparatus 501 and the thickness of the affected part of the patient (which may be a statistical value such as an average thickness) are stored in advance in the storage 225 of the information processing apparatus, and the projection image generating section 223 generates a projection image representing an image of the range of motion at the height using this information. In a case where the bed apparatus 501 can be driven such that the lying surface of the patient is inclined obliquely from a horizontal state, a projection image representing an image of a range of motion along the inclination of the bed apparatus 501 may be generated. In a case where information on the posture of the bed apparatus 501 is not stored in the storage 225, information on the posture of the bed apparatus 501 may be acquired by a method of measuring a distance to a marker installed in the bed apparatus. Alternatively, information on the posture of the bed apparatus 501 may be acquired by a method of communicating with a communication device provided in the bed apparatus to acquire at least one of the height and the inclination of the bed apparatus.

FIG. 5(B) is a plan view of another example in which an image is projected onto the range of motion of the distal end portion 111 by the projection device 141. An example in which an image 513B is projected onto the range of motion in a state where a patient is not lying on the bed apparatus 501 will be described. A projection image 513A includes the range of motion image 513B. By allowing the patient to lie on the bed apparatus such that, for example, the surgical site of the patient 502 is positioned in the displayed range of motion, the patient can be easily allowed to lie at an appropriate position. As an example, the range of motion is a range of motion in a plane at the height of the patient 502 or the height of the lying surface of the bed apparatus 501 in the actual three-dimensional range of motion of the distal end portion 111. In a case where the bed apparatus 501 can be driven such that the lying surface of the patient is inclined obliquely from a horizontal state, an image of a range of motion along the posture of the bed apparatus 501 may be generated and projected.

In a case where the image of the range of motion includes a plurality of types of regions, information for identifying the regions may be included in the image of the range of motion. For example, there are a region where the distal end portion 111 can be operated in a free posture and a region where the distal end portion 111 can be operated only in a specific posture. The identification information of each region may be color information.

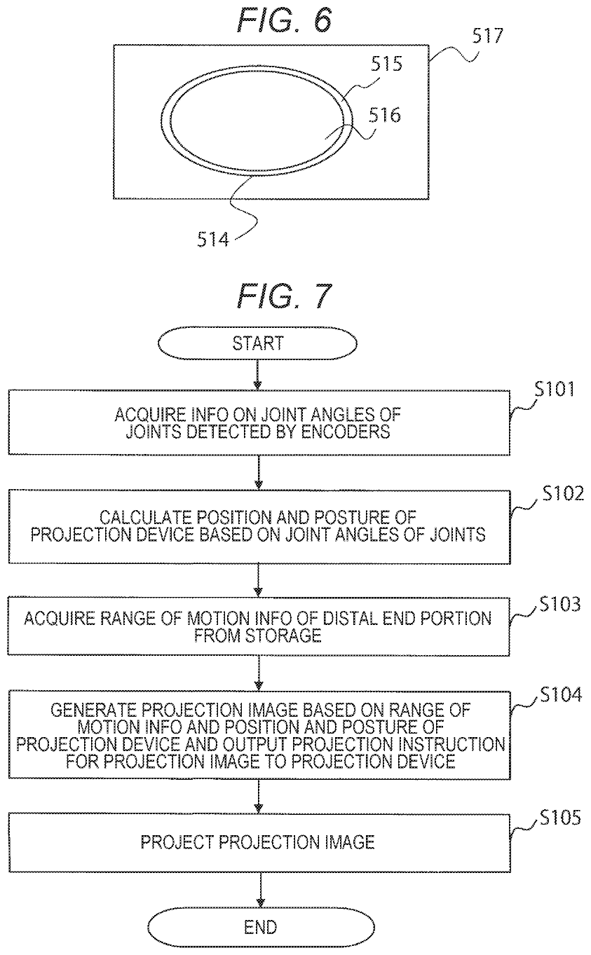

FIG. 6 illustrates an example in which a range of motion image 514 includes color information. A projection image 517 includes the range of motion image 514. The image 514 includes a plurality of regions 515 and 516. As an example, the region 516 is a region where the distal end portion 111 can be operated to the affected part in a free posture. The region 515 is a region where the distal end portion 111 can be operated to the affected part only in a specific posture (e.g., only in a direction perpendicular to the floor surface). By viewing a colored region, the user can determine a region where it is easy to operate with a medical arm and a region where it is difficult to operate with a medical arm. For a difficult region, it is conceivable to determine whether to operate by hand without using the medical arm, to rearrange the medical arm, or the like.

FIG. 7 is a flowchart of an example of operation of the information processing system 210 according to the present embodiment. As an example, the present operation is started in a case where a projection instruction for the motion space is input from the user via the input device 401 or in a case where the height of the arm 121 is changed after projection onto the motion space is performed. The present operation may be disclosed at other timings.

The joint angle acquiring section 221 acquires information on the joint angles (rotation angles) of the joints 123A to 123D from the encoders provided in the joints 123A to 123D (S101). Alternatively, the joint angle acquiring section 221 acquires information on the joint angles of the joints 123A to 123D stored in the storage 225 in advance.

The position and posture calculating section 222 calculates the position and posture of the projection device 141 on the basis of the joint angles (arm posture) of the joints 123A to 123D (S102). Specifically, the position and posture of the projection device 141 are calculated by forward kinematics on the basis of the joint angles of joints present from the base 131 to the installation location of the projection device 141.

The projection image generating section 223 acquires, from the storage 225, information expressing the range of motion of a target part (here, the distal end portion 111, etc.) of the robot 101 relative to the base 131 (S103). On the basis of the range of motion information of the distal end portion 111 relative to the base 131 and the position and posture of the projection device 141, the projection image generating section 223 generates a projection image that projects information specifying the range of motion of the distal end portion 111 onto the range of motion (S104). The output instruction section 224 outputs a projection instruction for the generated projection image to the projection device 141.

The projection device 141 projects the projection image in accordance with the projection instruction from the output instruction section 224 (S105). Therefore, an image for specifying the range of motion is displayed in the range of motion of the distal end portion 111.

The order of the steps illustrated in FIG. 7 is an example, and the order of some steps may be changed or a plurality of steps may be performed in parallel. For example, Step S103 may be performed before Step S101 or S102. Furthermore, Step S103 may be performed in parallel with Step S101 or S102.

As described above, even in a case where the user has changed the position of the arm, an image can be displayed in the range of motion by recalculating the posture of the arm and the position and posture of the projection device 141 following the change.

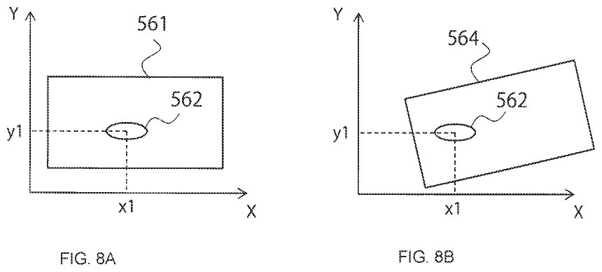

FIG. 8 illustrates an example in which an image is projected onto the range of motion even in a case where the posture of the arm has been changed. As illustrated in FIG. 8(A), a range of motion image 562 is illustrated in a projection image 561 at a position (x1, y1) of a two-dimensional coordinate system (XY coordinate system). When the posture of the arm is changed manually or by operating the input device 401, as illustrated in FIG. 8(B), the overall direction or shape of the projection image 564 is changed from that in FIG. 8(A), but the range of motion image 562 is displayed at the same position (x1, y1) and in the same direction. This is because the range of motion is in a fixed relationship with the base 131. In this manner, the range of motion information of the arm can be projected onto the target regardless of the posture of the arm.

In the example of FIG. 8, it is assumed that the relative position and the relative posture of the projection device 141 are the same with respect to the link 122D. However, depending on the position where the projection device 141 is installed or the posture of the arm, in a case where the posture of the arm is greatly changed, there may be a case where projection cannot be performed in the range of motion from the initial position and posture of the projection device 141. Also in this case, the image may be projected onto the range of motion by changing the relative position or the relative posture of the projection device 141 relative to the arm. The projection image generating section 223 may control the position and posture of the projection device 141 relative to the arm in this state.

According to the present embodiment as described above, the projection image that projects information specifying the range of motion onto the range of motion is generated on the basis of information regarding the range of motion of the target part of the robot and the posture of the arm calculated from the joint angle of the robot, and the projection image is projected from the projection device. Therefore, the user can intuitively understand the range of motion, and thus, in the operating room, the robot or the arm can be easily, appropriately, and quickly arranged so that the range of motion is at an appropriate position in accordance with the surgical information and the surgical situation held by the doctor. Also according to the present embodiment, the installability of the robot and the reliability of the installation position are improved.

(First Variation)

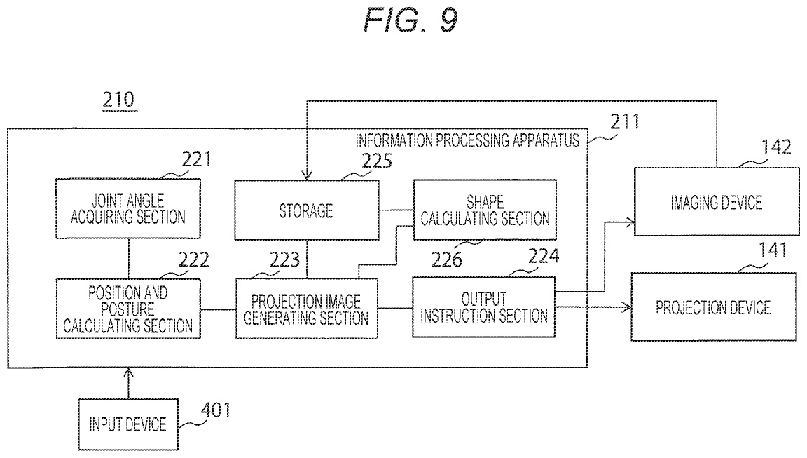

FIG. 9 is a block diagram of an information processing system according to a first variation. Blocks having the same names as those of the information processing system of the above-described embodiment are labeled with the same reference signs, and description thereof will be appropriately omitted except for extended or changed processing.

The information processing system 210 in FIG. 9 further includes at least one imaging device 142. The imaging device 142 is provided at an arbitrary location (part) of the arm 121 of the robot 101. For example, the imaging device 142 is provided in the distal end portion 111, an arbitrary joint, or an arbitrary link. The imaging device 142 includes a lens unit and an imaging element at a subsequent stage of the lens unit, observation light having passed through the lens unit is condensed on a light receiving surface of the imaging element, and an image signal is generated by photoelectric conversion. The imaging element is a complementary metal-oxide-semiconductor (CMOS) type image sensor, for example. Parameters such as magnification and focus of the imaging device can be adjusted by the control device 201.

In the first variation, the surface shape of the observation target (e.g., the observation part of the subject) is calculated using the imaging device 142 and the projection device 141. According to a projection instruction from the output instruction section 224, a two-dimensional image of a predetermined pattern is projected from the projection device 141 onto the observation target. The output instruction section 224 outputs an imaging instruction to the imaging device 142 so that the projected two-dimensional image is captured by the imaging device 142. The number of imaging devices 142 may be one or two or more. The imaging device 142 captures an image of the projected predetermined pattern, and stores the captured image data in the storage 225. A shape calculating section 226 specifies a correspondence relationship between the pattern of the projected image and the pattern included in the captured image, and calculates the surface shape of the observation target on the basis of the specified correspondence relationship and the principle of triangulation. That is, the depth at each position on the surface of the observation target is calculated. Calibration of the projection device 141 and the imaging device 142 may be performed in advance to acquire each piece of parameter information, and the parameter information may be used for calculation of the surface shape.

The projection image generating section 223 calculates the range of motion on the surface along the surface shape of the observation target in the three-dimensional range of motion of the target part (here, the distal end portion 111) of the robot 101. A projection image that projects information specifying the calculated range of motion onto the range of motion is generated. The output instruction section 224 outputs a projection instruction for the projection image to the projection device 141. As a result, the range of motion can be correctly displayed on the surface of the observation target. For example, in a case where there is unevenness on the surface of the observation target, there may be a position or region where the surgical site can be operated on from the distal end portion 111 and a position or region where the surgical site cannot be operated on depending on the position of the surface. In this case, in the present variation, an image is not projected on a position or region where operation cannot be performed correctly, and an image is projected only on a position or region where operation can be performed. In the above-described embodiment, an image of a range of motion in a plane at a certain height in a three-dimensional range of motion is projected. Therefore, in a case where an image is projected on an uneven observation target, the image can be projected even at a position where the distal end portion 111 cannot actually be operated (e.g., a recessed position where the distal end portion 111 does not reach). In the present variation, the range of motion can be more accurately displayed by generating a projection image based on measurement values of the surface shape of the observation target.

The projection image generating section 223 has calculated the range of motion on the surface of the observation target, but may calculate the range of motion (range of motion having a shape parallel to the shape of the surface of the observation target) at a height lower or higher than the surface of the observation target by a certain distance. For example, by displaying the range of motion at a height lower than the surface by a certain distance, the user (operator) can predict in advance the range of motion lower than the surface by the certain distance, so that surgery can be more appropriately performed. In addition, by displaying the range of motion at the height higher by the certain distance, for example, it is possible to appropriately grasp a region where the distal end portion 111 can be moved without making contact with the observation target.

In the present variation, the surface shape of the observation target has been calculated using the imaging device 142 and the projection device 141, but the surface shape may be calculated using a depth sensor such as a distance measuring sensor.

In the present variation, the surgical site of the patient is mainly assumed to be the observation target, but the lying surface of the patient on the bed apparatus, the floor surface on which the bed apparatus is installed during surgery, or the like may be used as the measurement target.

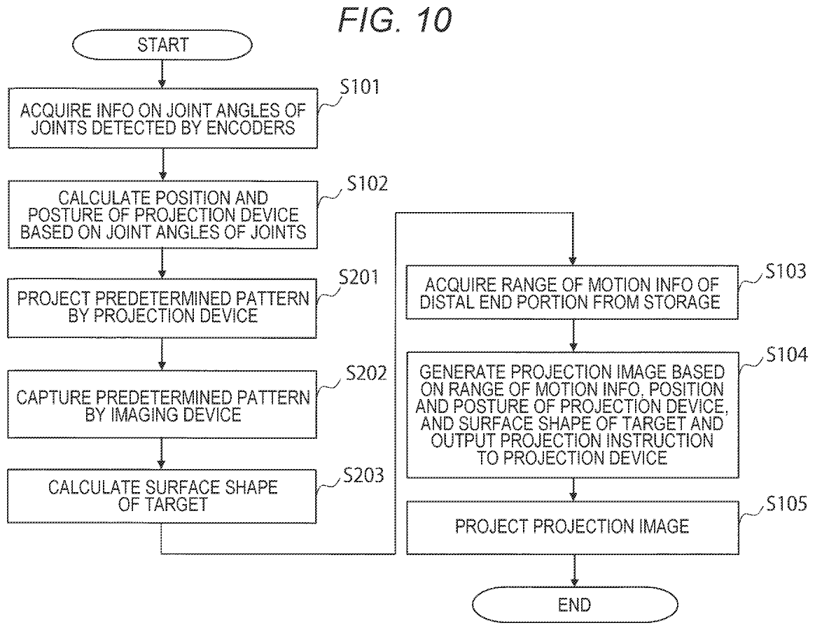

FIG. 10 is a flowchart of an example of operation of the information processing system according to the present variation. Steps S101 and S102 are the same as in the flowchart of FIG. 7 of the first embodiment described above.

After Step S102, according to an instruction of the output instruction section 224, an image of a predetermined pattern is projected from the projection device 141 onto the observation target (S201).

According to the instruction of the output instruction section 224, the imaging device 142 captures the image projected from the projection device 141 (S202).

A correspondence relationship between a predetermined pattern included in the projected image and a predetermined pattern included in the captured image is specified. The surface shape of the observation target is calculated using the principle of triangulation on the basis of the specified correspondence and the parameter information of the imaging device 142 and the projection device 141 acquired by advance calibration (S203).

The projection image generating section 223 acquires range of motion information of the distal end portion 111 from the storage 225 (S103). On the basis of the range of motion information of the distal end portion 111 and the surface shape of the observation target, the range of motion of the distal end portion 111 is specified on the surface of the observation target and a projection image that projects the information specifying the specified range of motion onto the range of motion is generated (S104). The output instruction section 224 outputs an instruction for generating the projection image to the projection device 141 (also S104). The projection device 141 projects the projection image in accordance with the instruction (S105).

The order of the steps in FIG. 10 is an example, and the order of some steps may be changed or a plurality of steps may be performed in parallel. For example, Steps S201 to S203 may be performed in parallel with Steps S101 and S102. Furthermore, Steps S201 to S203 may be performed before Step S101 or S102.

Information for identifying the distance (depth) by which the distal end portion 111 can move in the depth direction from the surface of the projection target (observation target) may be included in the image projected onto the observation target (e.g., the surgical site). The distance that can be moved in the depth direction from the surface is calculated by the shape calculating section 226 on the basis of the range of motion of the distal end portion 111 and the surface shape of the observation target.

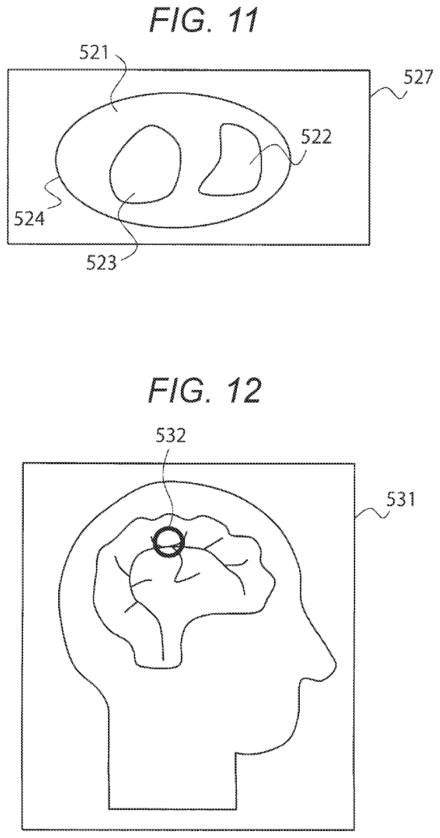

In FIG. 11, a range of motion image 524 includes information for identifying the distance that the distal end portion 111 can move in the depth direction (inward perpendicular direction along the paper surface). A projection image 527 includes the range of motion image 524. Each position in the image 524 is colored according to the size of the movable distance. A region 521 is a region in which the distal end portion 111 can move from the surface of the region 521 to the depth of a distance D1, and is given a first color (e.g., red). A region 522 is a region in which the distal end portion 111 can move from the surface of the region 521 to the depth of a distance D2 which is deeper than the distance D1, and is given a second color (e.g., yellow). A region 523 is a region in which the distal end portion 111 can move from the surface of the region 523 to the depth of a distance D3 which is deeper than the distance D2, and is given a third color (e.g., blue). The user can determine in advance how deep the distal end portion 111 of the robot can be operated by viewing each region identified by a color.

(Second Variation)

A block diagram of an information processing system of a second variation is the same as that of FIG. 3 of the above-described embodiment, and the function of the projection image generating section 223 is extended. In the present variation, a case where the above-described embodiment is extended is illustrated, but it is also possible to extend the function of the projection image generating section 223 of the first variation to realize a similar function to the present variation.

In the present variation, an image including a reference mark is acquired in advance. For example, an affected part image including an affected part (e.g., a tumor) of a patient is acquired by a technique such as computed tomography (CT) or magnetic resonance imaging (MRI) before surgery. The affected part image may be a two-dimensional image or a three-dimensional image. Using the range of motion information of the distal end portion 111, alignment between the affected part image and the range of motion is performed such that the affected part in the affected part image is included in the range of motion. The position of the affected part image associated with the range of motion of the robot relative to the base 131 is determined by this alignment. Alignment of the affected part image and the range of motion information may be manually performed by the user, or may be performed by the projection image generating section 223 or another computer. In a case where the projection image generating section 223 performs alignment, data of the affected part image is stored in the storage 225. As a method of alignment, for example, affected part detection may be performed by image analysis on the affected part image, and the range of motion information may be aligned with a detected affected part. The image analysis may be performed using a model such as a neural network generated by machine learning, or may be performed using image clustering or the like. Other methods may be used.

In an aligned state, the range of motion information is combined with the affected part image, and composite information (a composite image) in which the range of motion information is combined with the affected part image is generated. A projection image of the composite information is generated such that the range of motion information included in the composite information is displayed on the range of motion. The projection device 141 projects the projection image.

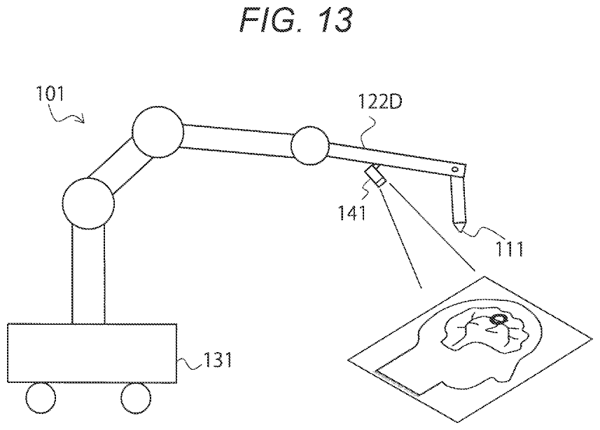

FIG. 12 illustrates an example in which range of motion information 532 is aligned with an affected part in an affected part image 531. A projection image that projects a composite image in which the range of motion information 532 is aligned with the affected part image 531 is generated such that the information 532 is displayed on the range of motion. The projection device 141 projects the projection image.

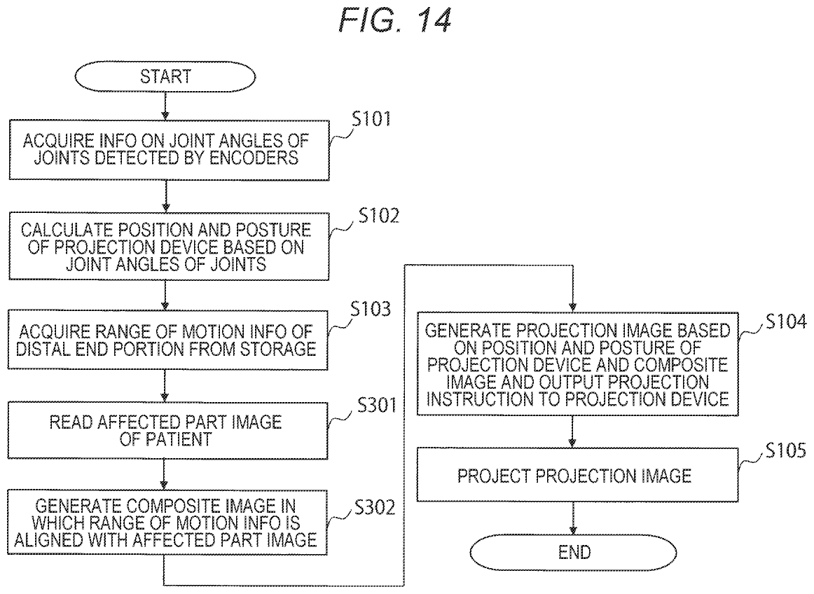

FIG. 13 illustrates an example in which an image is projected from the projection device 141 onto a floor surface of an operating room. By arranging the bed apparatus on which the patient is allowed to lie so as to align the affected part of the patient with the range of motion in the image projected on the bed apparatus, position adjustment between the robot 101 and the bed apparatus is facilitated. Instead of moving the bed apparatus, the position adjustment may be performed by moving the position of the robot 101.

Although the image is projected on the floor surface in FIG. 13, the image may be projected on the bed apparatus. In this case, the patient is allowed to lie on the bed apparatus such that the affected part of the patient is positioned in the range of motion in the image projected onto the bed apparatus. Therefore, the patient can be easily positioned in the bed apparatus.

In the above description, the range of motion information is aligned with the affected part of the patient as a reference mark. However, other than the affected part of the patient, items such as a mark affixed to the bed surface, an arbitrary part of the patient (head, waist), or a human form may be used.

FIG. 14 is a flowchart of an example of operation of the information processing system according to the present variation. In this example, the projection image generating section 223 aligns the affected part image with the range of motion information. Steps S101 to S103 are the same as in the flowchart of FIG. 7 of the first embodiment described above.

After Step S103, the projection image generating section 223 reads the affected part image from the storage 225 (S301) and generates a composite image in which the range of motion information acquired in Step S103 is aligned with the affected part in the affected part image (S302). On the basis of the position and posture of the projection device 141, a projection image that projects a composite image such that the range of motion information in the composite image is displayed in the range of motion is generated. The output instruction section 224 outputs an instruction for generating the projection image to the projection device 141 (S104). The imaging device 142 projects the projection image in accordance with the instruction from the output instruction section 224.

The order of the steps in FIG. 14 is an example, and the order of some steps may be changed or a plurality of steps may be performed in parallel. For example, Steps S103, S301, and S302 may be performed in parallel with Steps S101 and S102. Furthermore, Steps S103, S301, and S302 may be performed before Step S101 or S102.

(Third Variation)

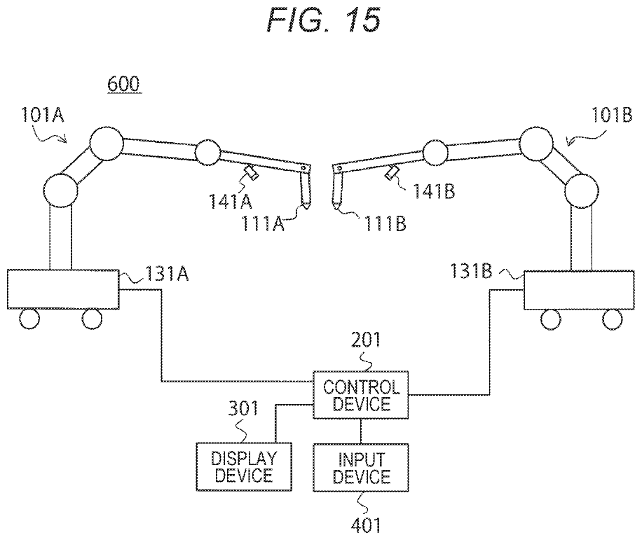

FIG. 15 is a diagram broadly illustrating an example of a surgical system 600 including an information processing system according to a third variation. The surgical system 100 includes a plurality of robots 101A and 101B, a control device 201, a display device 301, and an input device 401. The robots 101A and 101B have a similar configuration to the robot 101 in FIG. 1, and constituent elements of the robots are labeled with the same reference signs as those in FIG. 1, with different letters (A, B) added to the ends of the reference signs. Although two robots are illustrated in FIG. 15, the number of robots may be three or more. In the present variation, it is assumed that surgery is performed on a patient using a plurality of robots simultaneously.

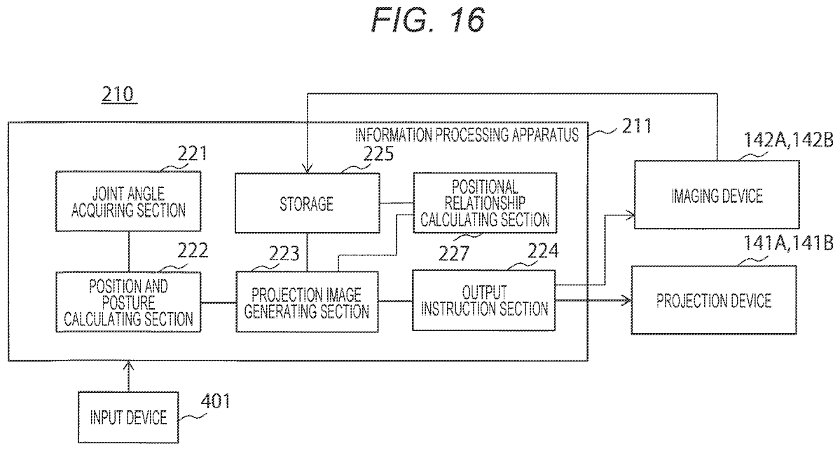

FIG. 16 is a block diagram of an information processing system according to the present variation. The information processing system 210 in FIG. 16 generates a projection image that projects information specifying an integrated region obtained by integrating ranges of motion of distal end portions 111A and 111B onto the integrated region, and projects the generated projection image. The information processing system 210 in FIG. 16 includes an information processing apparatus 211, an input device 401, imaging devices 142A and 142B, and projection devices 141A and 141B. The information processing apparatus 211 includes the same blocks 221 to 225 as described above in FIG. 9 and a positional relationship calculating section 227.

According to a projection instruction from the output instruction section 224, a predetermined pattern image (correction image) is projected from the projection device 141A or 142B. As an example, the projection target is a floor surface or a bed surface. The output instruction section 224 outputs an imaging instruction to the imaging devices 142A and 142B so that the projected correction image is captured by the imaging devices 142A and 142B. The imaging devices 142A and 142B capture both correction images projected from the two projection devices in a posture, and store the captured image data in the storage 225.

The positional relationship calculating section 227 calculates a positional relationship (arm positional relationship) between the two robots 101A and 101B on the basis of the image data captured by the imaging devices 142A and 142B. For example, the position of the projected pattern is determined by the principle of triangulation from the relationship between the projected pattern by the projection device 141A and the projected pattern captured by the projection device 142A. Since the positional relationship between the imaging devices is obtained by capturing images of the projected patterns by the imaging device 142A and the imaging device 142B, the positional relationship between the bases of the robots can be obtained. In addition, a model (e.g., a neural network) that uses two pieces of image data as inputs and outputs the positional relationship between the two robots may be learned in advance, and the positional relationship may be calculated using the model.

The projection image generating section 223 calculates an integrated region in which the ranges of motion of the distal end portions 111A and 111B are integrated on the basis of the information regarding the ranges of motion of the distal end portions 111A and 111B, the positions and the postures of the projection devices 141A and 141B, and the positional relationship calculated by the positional relationship calculating section 227. A projection image that projects information specifying the calculated integrated region onto the integrated region is generated. The output instruction section outputs a projection instruction for the generated projection image to the projection device 141A or 141B.

In addition, the projection image generating section 223 may specify a region where interference between the two distal end portions 111 is likely to occur in the integrated region on the basis of the above positional relationship, and include information for identifying the specified region in the image. Specifically, a region (first region) in which interference is likely to occur between the distal end portions 111, a region (second region) where the two distal end portions 111 are simultaneously movable and interference is unlikely to occur, and a region (third region) where only the distal end portions 111 are movable may be specified, and information for identifying these three regions may be included in the image. For example, a region having a constant width from the center of an intersection region of the two regions is referred to as a first region, a region other than the first region in the intersection region is referred to as a second region, and a region other to these is referred to as a third region.

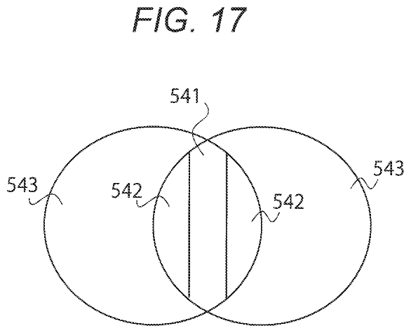

FIG. 17 illustrates an example in which an image is projected onto the integrated region. The image includes a region 543 (third region) where only the distal end portions 111A and 111B are individually movable, a region 542 (second region) where the distal end portions 111A and 111B are simultaneously movable and interference is unlikely to occur, and a region 541 (first region) where interference between the distal end portions 111A and 111B is likely to occur. Interference refers to a collision between the distal end portions or failure to simultaneously operate on the same object, for example. The regions 541 to 543 may be displayed in different colors or patterns. The user may confirm the integrated region directly or through the display device 301 to readjust the arrangement between the robots or the arms. Therefore, the size of each region can be adjusted, for example, by increasing the size of the region 542.

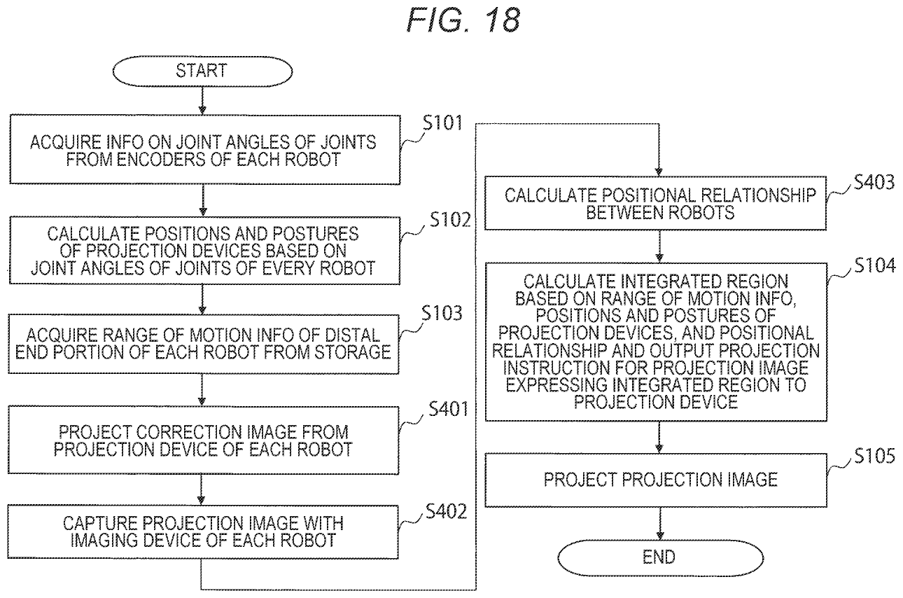

FIG. 18 is a flowchart of an example of operation of the information processing system 210 according to the present variation.

The joint angle acquiring section 221 acquires information on the joint angles (rotation angles) of each joint from the encoders provided in the joints of the robots 101A and 101B (S101). The position and posture calculating section 222 calculates the position and posture of the projection devices 141A and 141B on the basis of the joint angles of the joints of the robots 101A and 101B (S102). In addition to the positions and postures of the projection devices 141A and 141B, the positions and postures of the imaging devices 142A and 142B may be calculated. The projection image generating section 223 acquires, from the storage 225, information expressing the ranges of motion of target parts (distal end portions 111, etc.) of the robots 101A and 101B relative to the bases 131A and 131B (S103).

The projection image generating section 223 generates projection images representing correction images for the robots 101A and 101B, respectively (S401). The output instruction section 224 outputs a projection instruction for the correction images represented by the projection images to the projection devices 141A and 141B of the robots 101A and 101B (also Step S401).

The output instruction section 224 outputs an imaging instruction to the imaging devices 142A and 142B of the robots 101A and 101B (S402). The imaging devices 142A and 142B perform imaging and provide captured image data to the information processing apparatus 211 (also S402). The information processing apparatus 211 stores each piece of correction image data in the storage 225 (also Step S402). Each piece of correction image data includes correction images projected from both projection devices 141A and 141B.

The positional relationship calculating section 227 calculates a positional relationship (arm positional relationship) between the two robots on the basis of the correction image data captured by the imaging devices 142A and 142B (S403).

The projection image generating section 223 calculates an integrated region in which the ranges of motion of the distal end portions 111A and 111B are integrated on the basis of the information regarding the ranges of motion of the distal end portions 111A and 111B, the positions and postures of the projection devices 141A and 141B, and the positional relationship calculated by the positional relationship calculating section 227 (S104). As an example, the integrated region includes a region (first region) where only the distal end portions 111A and 111B are individually movable, a region (second region) where the distal end portions 111A and 111B are simultaneously movable and interference is unlikely to occur, and a region (third region) where interference between the distal end portions 111A and 111B is likely to occur. The projection image generating section 223 generates a projection image that projects information specifying the integrated region onto the integrated region (also S104). The output instruction section 224 outputs a projection instruction for the projection image to the projection device 141A or 141B (also S104).

The projection device 141A or 141B projects the projection image in accordance with the projection instruction from the output instruction section 224 (S105).

In the present variation, the positional relationship between the robots is calculated using the imaging devices 142A and 142B and the projection devices 141A and 141B. However, in a case where each robot includes a position detection function, the information processing apparatus 211 may communicate with each robot to acquire position information of each robot. The positional relationship calculating section 227 calculates a positional relationship between the robots on the basis of the positional information of each robot.

Note that the above-described embodiments illustrate examples for embodying the present disclosure, and the present disclosure can be implemented in various other forms. For example, various modifications, substitutions, omissions, or combinations thereof can be made without departing from the gist of the present disclosure. Such modifications, substitutions, omissions, and the like are also included in the scope of the present disclosure and are included in the invention described in the claims and the equivalent scope thereof.

Furthermore, the effects of the present disclosure described in the present specification are merely examples, and other effects may be provided.

The present disclosure can also have the following configurations.

### [Item 1]

An information processing apparatus including:

a projection image generating section which generates a projection image that projects information specifying a range of motion of a target part of a medical arm onto the range of motion on the basis of first information regarding the range of motion and second information regarding a position and a posture of a projection device that projects an image in an operating room; and

an output instruction section which outputs a projection instruction for the projection image to the projection device.

### [Item 2]

The information processing apparatus according to item 1, in which

the range of motion of the target part includes a region in which a distal end portion of the medical arm is movable or a region in which an imaging device provided at an arbitrary part of the medical arm is capable of imaging.

### [Item 3]

The information processing apparatus according to item 1 or 2, further including a position and posture calculating section, in which

the projection device is provided in the medical arm, and

the position and posture calculating section calculates the position and the posture of the projection device on the basis of the posture of the medical arm.

### [Item 4]

The information processing apparatus according to any one of items 1 to 3, in which

the projection image generating section generates the projection image on the basis of at least one of a position and a posture of a target on which the projection image is projected.

### [Item 5]

The information processing apparatus according to item 4, in which

the target on which the projection image is projected is a lying surface of a bed apparatus on which a subject to undergo medical treatment is allowed to lie, an observation part of the subject allowed to lie on the bed apparatus, or a floor surface on which the bed apparatus is installed.

### [Item 6]

The information processing apparatus according to item 4, further including

a shape calculating section which calculates a surface shape of the target on which the projection image is projected, in which

the projection image generating section generates the projection image on the basis of the surface shape.

### [Item 7]

The information processing apparatus according to item 6, in which

the range of motion is a range of motion on a surface of the target on which the projection image is projected.

### [Item 8]

The information processing apparatus according to item 7, in which

the range of motion is a range of motion at a height lower or higher than the surface of the target by a certain distance.

### [Item 9]

The information processing apparatus according to item 8, in which

the range of motion having the height higher by the certain distance is a region where the target part is movable without making contact with the target.

### [Item 10]

The information processing apparatus according to item 6, in which

the projection image includes information for identifying a movable distance in a depth direction of the target on which the projection image is projected.

### [Item 11]

The information processing apparatus according to any one of items 1 to 10, in which

the projection device is a three-dimensional projection device, and

the projection image is a three-dimensional image.

### [Item 12]

The information processing apparatus according to any one of items 1 to 11, in which

the projection image generating section generates composite information obtained by combining information on the range of motion in alignment with a reference mark of an image including the reference mark, and generates the projection image on which the composite information is projected.

### [Item 13]

The information processing apparatus according to item 12, in which,

the reference mark is an affected part of the subject in an image including the affected part.

### [Item 14]

The information processing apparatus according to any one of items 1 to 13, in which

the projection image generating section calculates an integrated region obtained by integrating ranges of motion of target parts of a plurality of medical arms on the basis of a plurality of the first information regarding the ranges of motion of the target parts of the plurality of medical arms and the second information, and generates the projection image that projects information for specifying the integrated region onto the integrated region.

### [Item 15]

The information processing apparatus according to item 14, in which

the integrated region includes a first region in which the plurality of medical arms interfere with each other, and

the projection image includes information for identifying the first region.

### [Item 16]

The information processing apparatus according to item 15, in which

a second region different from the first region in the integrated region is different in color from the first region.

### [Item 17]

The information processing apparatus according to item 14, in which

the projection image generating section generates the projection image on the basis of the positional relationship of the plurality of medical arms.

### [Item 18]

The information processing apparatus according to item 17, further including a positional relationship calculating section, in which

the projection device is provided in a plurality thereof

the projection devices are installed in the plurality of medical arms,

correction images including predetermined patterns are projected from the projection devices of the medical arms, and

the positional relationship calculating section acquires image data obtained by capturing the plurality of projected correction images from the plurality of imaging devices, and calculates a positional relationship between the plurality of medical arms on the basis of the plurality of predetermined patterns included in each piece of the acquired image data.

### [Item 19]

An information processing system including:

a projection device which projects an image in an operating room;

a projection image generating section which generates a projection image that projects information specifying a range of motion of a target part of a medical arm onto the range of motion on the basis of first information regarding the range of motion and second information regarding a position and a posture of the projection device; and

an output instruction section which outputs a projection instruction for the projection image to the projection device.

### [Item 20]

An information processing method including:

generating a projection image that projects information specifying a range of motion of a target part of a medical arm onto the range of motion on the basis of first information regarding the range of motion and second information regarding a position and a posture of a projection device that projects an image in an operating room; and

projecting the projection image by the projection device.

### REFERENCE SIGNS LIST

100 Surgical system
101 Robot
201 Control device
301 Display device
401 Input device
111 Distal end portion
121 Arm
131 Base
122A to 122E Link
501 Bed apparatus
502 Patient
210 Information processing system
211 Information processing apparatus
141 Projection device
401 Input device
221 Joint angle acquiring section
222 Position and posture calculating section
223 Projection image generating section
224 Output instruction section
225 Storage
226 Shape calculating section
227 Positional relationship calculating section

1. An information processing apparatus comprising:
a projection image generating section configured to generate a projection image that projects information specifying a range of motion of a target part of a medical arm onto the range of motion on a basis of first information regarding the range of motion and second information regarding a position and a posture of a projection device that projects an image in an operating room; and
an output instruction section configured to output a projection instruction for the projection image to the projection device.

2. The information processing apparatus according to claim 1, wherein
the range of motion of the target part includes a region in which a distal end portion of the medical arm is movable or a region in which an imaging device provided at an arbitrary part of the medical arm is capable of imaging.

3. The information processing apparatus according to claim 1, further comprising a position and posture calculating section, wherein
the projection device is provided in the medical arm, and
the position and posture calculating section calculates the position and the posture of the projection device on a basis of the posture of the medical arm.

4. The information processing apparatus according to claim 1, wherein
the projection image generating section generates the projection image on a basis of at least one of a position and a posture of a target on which the projection image is projected.

5. The information processing apparatus according to claim 4, wherein
the target on which the projection image is projected is a lying surface of a bed apparatus on which a subject to undergo medical treatment is allowed to lie, an observation part of the subject allowed to lie on the bed apparatus, or a floor surface on which the bed apparatus is installed.

6. The information processing apparatus according to claim 4, further comprising
a shape calculating section configured to calculate a surface shape of the target on which the projection image is projected, wherein
the projection image generating section generates the projection image on a basis of the surface shape.

7. The information processing apparatus according to claim 6, wherein
the range of motion is a range of motion on a surface of the target on which the projection image is projected.

8. The information processing apparatus according to claim 7, wherein
the range of motion is a range of motion at a height lower or higher than the surface of the target by a certain distance.

9. The information processing apparatus according to claim 8, wherein
the range of motion at the height higher by the certain distance is a region where the target part is movable without making contact with the target.

10. The information processing apparatus according to claim 6, wherein
the projection image includes information for identifying a movable distance in a depth direction of the target on which the projection image is projected.

11. The information processing apparatus according to claim 1, wherein
the projection device is a three-dimensional projection device, and
the projection image is a three-dimensional image.

12. The information processing apparatus according to claim 1, wherein
the projection image generating section generates composite information obtained by combining information on the range of motion in alignment with a reference mark of an image including the reference mark, and generates the projection image on which the composite information is projected.

13. The information processing apparatus according to claim 12, wherein,
the reference mark is an affected part of the subject in an image including the affected part.

14. The information processing apparatus according to claim 1, wherein
the projection image generating section calculates an integrated region obtained by integrating ranges of motion of target parts of a plurality of medical arms on a basis of a plurality of the first information regarding the ranges of motion of the target parts of the plurality of medical arms and the second information, and generates the projection image that projects information for specifying the integrated region onto the integrated region.

15. The information processing apparatus according to claim 14, wherein
the integrated region includes a first region in which the plurality of medical arms interfere with each other, and
the projection image includes information for identifying the first region.

16. The information processing apparatus according to claim 15, wherein
a second region different from the first region in the integrated region is different in color from the first region.

17. The information processing apparatus according to claim 14, wherein
the projection image generating section generates the projection image on a basis of the positional relationship of the plurality of medical arms.

18. The information processing apparatus according to claim 17, further comprising a positional relationship calculating section, wherein
the projection device is provided in a plurality thereof,
the projection devices are installed in the plurality of medical arms,
correction images including predetermined patterns are projected from the projection devices of the medical arms, and
the positional relationship calculating section acquires image data obtained by capturing the plurality of projected correction images from the plurality of imaging devices, and calculates a positional relationship between the plurality of medical arms on a basis of the plurality of predetermined patterns included in each piece of the acquired image data.

19. An information processing system comprising:
a projection device configured to project an image in an operating room;
a projection image generating section configured to generate a projection image that projects information specifying a range of motion of a target part of a medical arm onto the range of motion on a basis of first information regarding the range of motion and second information regarding a position and a posture of the projection device; and
an output instruction section configured to output a projection instruction for the projection image to the projection device.

20. An information processing method comprising:
generating a projection image that projects information specifying a range of motion of a target part of a medical arm onto the range of motion on a basis of first information regarding the range of motion and second information regarding a position and a posture of a projection device that projects an image in an operating room; and
projecting the projection image by the projection device.
